(19) United States
(12) Patent Application Publication
(10) Pub. No.: US 2018/0289874 A1
(43) Pub. Date: Oct. 11, 2018

(54) Apparatus for Improving Experience of Breastfeeding

(71) Applicant: Sean Xiaolu Wang, Wilmington, DE (US)

(72) Inventor: Sean Xiaolu Wang, Wilmington, DE (US)

(73) Assignee: BWT Property, Inc., Newark, DE (US)

(21) Appl. No.: 15/925,012

(22) Filed: Mar. 19, 2018

Related U.S. Application Data
(60) Provisional application No. 62/482,862, filed on Apr. 7, 2017.

(51) Int. Cl. A61M 1/06; A61N 5/06

(52) U.S. Cl. CPC A61M 1/062; A61N 5/0613; A61M 2205/052; A61M 2205/053; A61N 2005/0645; A61N 2005/0649; A61N 2005/067; A61N 2005/0653; A61N 2005/0654; A61N 2005/0659; A61N 2005/0661; A61N 2005/0663; A61N 2005/0652

(57) ABSTRACT

The photo therapy apparatus of the present invention comprises one or more light sources which are integrated with a breast pump for providing photo therapy treatment to the breast and nipple of women during pumping of breast milk. The light sources can be lasers, light emitting diodes (LEDs), lamps, or any other types of light sources which produce therapeutic light in a desired wavelength range. The therapeutic light is delivered through the breast-shield or flange of the breast pump to the breast and nipple and produces a variety of photochemical processes, e.g. increase of ATP (adenosine triphosphate), triggering of photo neurological response and activation of enzymes, changes in local pressure, increases in temperature, and deformation of cellular membranes. Such photochemical reaction helps to reduce pain and promote healing of nipple lesion and in the meantime enhance yield of breast milk.

### REFERENCE TO RELATED APPLICATIONS

This application claims inventions which were disclosed in Provisional Patent Application No. 62/482,862, filed Apr. 7, 2017, entitled “APPARATUS FOR IMPROVING EXPERIENCE OF BREASTFEEDING”. The benefit under 35 USC § 119(e) of the above mentioned United States Provisional Application is hereby claimed, and the aforementioned application is hereby incorporated herein by reference.

### FIELD OF THE INVENTION

This invention generally relates to a photo therapy apparatus, and more specifically to a photo therapy apparatus for improving experience of breastfeeding.

### BACKGROUND OF THE INVENTION

Pain accruing from nipple lesions caused by inadequate latching by the baby is a common complaint among breastfeeding women and an important obstacle to successful breastfeeding. Low-level laser therapy was considered effective for treating nipple lesions in breastfeeding women with pain, providing relief and prolonging exclusive breastfeeding. It is the thus goal of the present invention to provide a convenient photo therapy apparatus for improving experience of breastfeeding. The photo therapy apparatus comprises one or more light sources that are integrated with a breast pump for reduction of sore and inflammation, treatment of damaged nipples, blocked ducts, and mastitis, which in turn bring in the advantage of more milk production, more milk yielded per session, and more comfort with reduced pump pressure.

### SUMMARY OF THE INVENTION

The photo therapy apparatus of the present invention comprises one or more light sources which are integrated with a breast pump for providing photo therapy treatment to the breast and nipple of women during pumping of breast milk. The light sources can be lasers, light emitting diodes (LEDs), lamps, or any other types of light sources which produce therapeutic light in a desired wavelength range. The therapeutic light is delivered through the breast-shield or flange of the breast pump to the breast and nipple and produces a variety of photochemical processes, e.g. increase of ATP (adenosine triphosphate), triggering of photo neurological response and activation of enzymes, changes in local pressure, increases in temperature, and deformation of cellular membranes. Such photochemical reaction helps to reduce pain and promote healing of nipple lesion and in the meantime enhance yield of breast milk.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a first exemplary embodiment of the photo therapy apparatus for improving experience of breastfeeding; and

FIG. 2 illustrates a second exemplary embodiment of the photo therapy apparatus for improving experience of breastfeeding.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and apparatus components related to a photo therapy apparatus for improving experience of breastfeeding. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

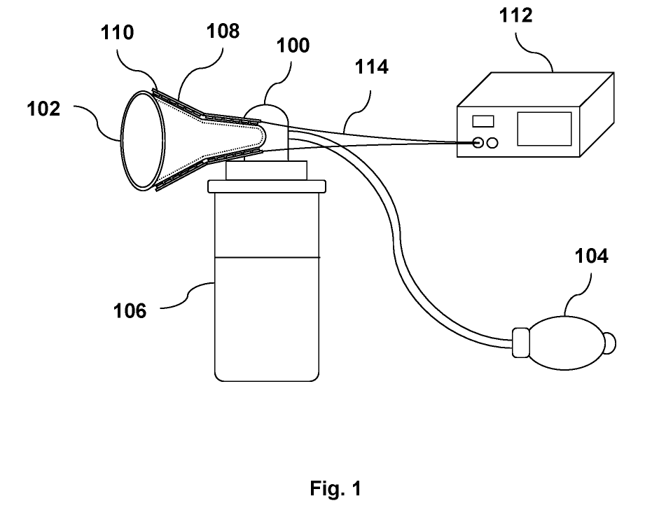

A first exemplary embodiment of the photo therapy apparatus is shown in FIG. 1. The photo therapy apparatus comprises one or more light sources 108 which are mounted on the back of a breast-shield or flange 102 of a breast pump 100. The breast pump 100 is a mechanical device that extracts milk from the breasts of a lactating woman by using a suction action produced by a pump 104 to pull the nipple into the tunnel of the breast-shield or flange 102 and extract breast milk into a container 106. Breast pumps may be either manually powered or electrically powered. Many women use breast pumps to express breast milk, which is later bottle fed to their child by a caregiver. The breast pump may also be used to stimulate lactation for women with a low milk supply or those who have not just given birth. The light source 108 preferably consists of lasers such as laser diodes, diode pumped solid state lasers (DPSSLs), or light emitting diodes (LEDs), organic light emitting diode (OLED) sheets, and electrical luminance (EL) emitters 110, which produce therapeutic light in a desired wavelength range. The therapeutic light is then delivered through the breast-shield or flange 102 of the breast pump 100 to the breast and nipple and produces a variety of photochemical processes, e.g. increase of ATP (adenosine triphosphate), triggering of photo neurological response and activation of enzymes, changes in local pressure, increases in temperature, and deformation of cellular membranes. Such photochemical reaction helps to reduce pain and promote healing of nipple lesion and in the meantime enhance yield of breast milk. The light source 108 may also comprise lamp light sources or other types of broadband light sources such as supercontinuum light sources which are integrated with optical filters to select a desired output wavelength range. The light source 108 may be pulsed to enhance its treatment effect. The output power, duration, and pulsation property (i.e. pulse width, repetition rate, duty cycle) of the light source 108 is controlled by a controller 112 via wired connection 114 or wireless connection to adjust the light dosage applied to the breast tissue. Such properties of the light source 108 may be optimized in accordance to the skin color, skin thickness, and age of the women to produce the best therapeutic effect.

Depending on the output wavelength of the light source 108, the therapeutic light can provide a variety of treatment effects. The ultraviolet (UV)-blue light, at a wavelength of 370 to 490 nm, has high photon energy, which can help to produce singlet oxygen and effectively destroy bacteria. The red light, at a wavelength of 620 to 700 nm, can penetrate human tissue to a depth of about 8-10 mm. Skin layers, because of their high blood and water content, easily absorb red light, which helps to increase blood circulation and decrease inflammation/irritation. The near-infrared (NIR) light, at a wavelength of >700 nm, has been demonstrated to be beneficial for increasing cytochrome oxidase activity and ATP (adenosine triphosphate) content as well as promoting wound healing and relieving pain. In addition, the near-infrared light penetrates to a depth of about 30-40 mm, which makes it more effective for deep muscles. The light source 108 may comprise multiple light sources with different output wavelengths, each wavelength matching with the absorption band of a specific chromophore (water, hemoglobin, lipid, protein, etc.) of the subject breast tissue.

In a slight variation of the present embodiment, an optical component (not shown) is inserted between the light source 108 and the breast-shield or flange 102 for controlling the intensity distribution of the therapeutic light. Such optical component can be a holographic diffuser or a micro-lens array which has a large number of microstructures on its surface. As one example, the holographic diffuser or the micro-lens array may produce an unevenly distributed light intensity. This intensity modulation will help to further improve the therapeutic effect of the light source 108. In yet another variation of the present embodiment, the light source 108 may be embedded inside the breast-shield or flange 102 of a breast pump 100 to produce therapeutic light thereof.

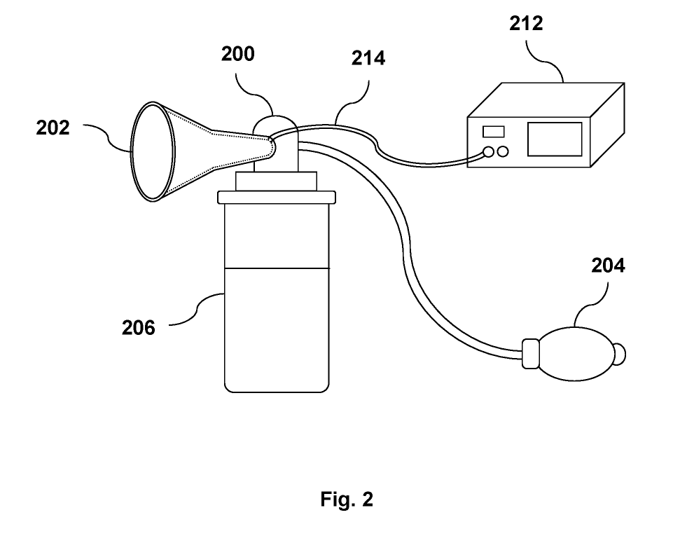

FIG. 2 illustrates a second exemplary embodiment of the photo therapy apparatus. Here the therapeutic light is produced by a light source 212 and delivered by a light guide (such as an optical fiber) 214 into the breast-shield or flange 202 of a breast pump 200. The breast-shield or flange 202, which is made of a translucent material, serves as a side emitting light guide that scatters the therapeutic light into the breast tissue to provide photo therapy treatment. The translucent material may comprise micro-sized particles and micro V-grooves or other types of micro-structures for increasing the scattering of the therapeutic light.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A photo therapy apparatus for improving experience of breastfeeding for a woman, the photo therapy apparatus comprising:
a breast pump for extracting breast milk from breasts of the woman; and
a light source for producing therapeutic light;
wherein the therapeutic light is delivered through a breast-shield or flange of the breast pump to the breasts of the woman for providing photo therapy treatment.

2. The photo therapy apparatus of claim 1, wherein the light source is embedded inside the breast-shield or flange of the breast pump.

3. The photo therapy apparatus of claim 1, wherein the therapeutic light is delivered through a light guide into the breast-shield or flange of the breast pump and is scattered by the breast-shield or flange to the breasts of the woman.

4. The photo therapy apparatus of claim 1, further comprising an optical component for controlling an intensity distribution of the therapeutic light.

5. The photo therapy apparatus of claim 1, wherein the light source comprises lasers.

6. The photo therapy apparatus of claim 1, wherein the light source comprises light emitting diodes (LEDs).

7. The photo therapy apparatus of claim 1, wherein the light source comprises organic light emitting diodes (OLEDs).

8. The photo therapy apparatus of claim 1, wherein the light source comprises electrical luminance (EL) emitters.

9. The photo therapy apparatus of claim 1, wherein the light source comprises lamp light sources.

10. The photo therapy apparatus of claim 1, wherein the light source comprises supercontinuum light sources.

11. The photo therapy apparatus of claim 1, wherein the light source comprises optical filters for selecting a desired output wavelength range.

12. A method for improving experience of breastfeeding for a woman, the method comprising the steps of:
providing a breast pump for extracting breast milk from breasts of the woman;
providing a light source for producing therapeutic light; and
delivering the therapeutic light through a breast-shield or flange of the breast pump to the breasts of the woman for providing photo therapy treatment.

13. The method of claim 12, wherein the light source is embedded inside the breast-shield or flange of the breast pump.

14. The method of claim 12, wherein the therapeutic light is delivered through a light guide into the breast-shield or flange of the breast pump and is scattered by the breast-shield or flange to the breasts of the woman.

15. The method of claim 12, further comprising a step of providing an optical component for controlling an intensity distribution of the therapeutic light.

16. The method of claim 12, wherein the light source comprises lasers.

17. The method of claim 12, wherein the light source comprises light emitting diodes (LEDs).

18. The method of claim 12, wherein the light source comprises lamp light sources.

19. The method of claim 12, wherein the light source comprises supercontinuum light sources.

20. The method of claim 12, wherein the light source comprises optical filters for selecting a desired output wavelength range.
